(19) United States
(12) Patent Application Publication
(10) Pub. No.: US 2019/0046466 A1
(43) Pub. Date: Feb. 14, 2019

(54) COMPOSITION AND METHOD FOR INHIBITING PLATELET AGGREGATION

(71) Applicant: Paul A. Knepper, Chicago, IL (US)

(72) Inventor: Paul A. Knepper, Chicago, IL (US)

(21) Appl. No.: 15/752,458

(22) PCT Filed: Aug. 18, 2016
(86) PCT No.: PCT/US2016/047524

§ 371 Date: Feb. 13, 2018

Related U.S. Application Data
(60) Provisional application No. 62/207,535, filed on Aug. 20, 2015.

(51) Int. Cl. A61K 31/05; A61K 9/48; A61K 31/352; A61K 31/485; A61P 25/28

(52) U.S. Cl. CPC A61K 31/05; A61K 9/4858; A61P 25/28; A61K 31/485; A61K 31/352

(57) ABSTRACT

Pharmaceutical compositions containing a stilbene such as resveratrol, a flavonol such as quercetin, and a μ-opioid receptor antagonist such as naltrexone inhibit aggregation of superactivated platelets, block activation of the coagulation cascade, and are useful for treating microvascular diseases including neurodegenerative diseases such as Alzheimer's disease and dementia, for treating primary open-angle glaucoma, for reducing scar formation, and like afflictions that involve the coagulation cascade.

### CROSS-REFERENCE TO RELATED APPLICATION

This application claims benefit of U.S. Provisional Application Ser. No. 62/207,535, filed on 20 Aug. 2015, the entire disclosure of which is incorporated herein by reference.

### FIELD OF INVENTION

This invention relates to therapy involving inhibition of undesirable activation of platelets.

### BACKGROUND OF THE INVENTION

Microvascular diseases account for more than one-third of human deaths worldwide. A wide number of microvascular diseases exhibit abnormal platelet functions. Platelets play an important role in arresting of bleeding, i.e., clotting. If platelets do not function as they should, obstructive clotting or serious bleeding can occur. Table 1, below, provides a sampling of conditions, and impacted populations ranging from the widespread to the less common, in which platelet function is abnormal.

TABLE 1
Prevalence and Incidence of Microvascular Diseases with
Abnormal Platelet Function in the U.S. and Worldwide

| | U.S. | | | Worldwide | | |
|---|---|---|---|---|---|---|
| Condition | Prevalence | | Incidence | Prevalence | | Incidence |
| Alzheimer's disease | 5.4 | million | 476,000 | 47 | million | |
| Arthritis | 52.5 | million | | | | |
| Osteoarthritis | 30.8 | million | | | | |
| Rheumatoid arthritis | 1.5 | million | | 94.9 | million† | 2,993 million‡ |
| Cardiovascular disease | 85.6 | million | | | | |
| Deep vein thrombosis | 600,000/year | | 377,676* | 10 | million | |
| Pulmonary embolism | | | | | | |
| Diabetes | 29.1 | million | 1.4 million | 415 | million | |
| Fibromyalgia | 10 | million | | 438 | million§ | |
| Glaucoma | 3 | million | | 60.5 | million | |
| Open-angle glaucoma | 2.7 | million | | | | |
| POAG | | | | 57.5 | million | |
| Heart disease | 27.6 | million | | | | |
| Lupus | 1.5 | million | | 5 | million | |
| Myocardial infarction | 735,000/year | | 635,000 | | | |
| Scleroderma | 300,000 | | 7,747** | 2.5 | million | |
| Systemic scleroderma | 75,000 | | 6,456*** | | | |
| Sjögren's Syndrome | 4 | million | | | | |

| | | | | |
|---|---|---|---|---|
| Stroke | 795,000 | 610,000 | 33 | million |

*Based on an incidence rate of 117 cases per 100,000 individuals and 2016 U.S. population estimate of 322.8 million.
**Based on an incidence rate of 24 cases per 1 million individuals and 2016 U.S. population estimate of 322.8 million.
***Based on an incidence rate of 2 cases per 1 million individuals and 2016 U.S. population estimate of 322.8 million.
†Based on a prevalence of 1.3% and rounded 2016 world population estimate of 7.3 billion.
‡Based on an incidence rate of 41 cases per 100,000 individuals and 2016 world population estimate of 7.3 billion.
§Based on a prevalence of 6% and 2016 world population estimate of 7.3 billion.

Platelets (thrombocytes) are fragments of cytoplasm whose primary function is to arrest bleeding. In the blood stream, platelets travel singly, as smooth-surfaced discs. When blood vessels suffer trauma, however, platelets adhere to the exposed subendothelial fibrils, become sticky, and adhere to one another to form a hemostatic plug. The coagulation cascade is illustrated in FIG. 1. Toll-like receptor 4 (TLR4) is a key innate immune receptor in the coagulation cascade, recognizes damage activated molecular patterns, and is important in all microvascular diseases.

A separate and discrete subpopulation of platelets exhibiting enhanced procoagulant (prothrombogenic) activity after stimulation with strong agonists has been identified. See, for example, Mazepe et al., Arterioscler Thromb Vasc Biol 33:1747-1752 (2013). This subpopulation of platelets is referred to as superactivated platelets (SAPs). SAPs are prothrombogenic platelets that are elevated in patients suffering from microvascular diseases such as primary open-angle glaucoma (POAG), a neurodegenerative disease, diabetes, etc.

Individuals afflicted with such microvascular conditions as Alzheimer's disease, glaucoma, connective tissue disorders, and autoimmune disease benefit from drugs that impact the abnormal platelet function, hemorrhaging, and/or thrombotic events associated with these conditions.

The present invention provides a composition and method for ameliorating the effects of SAPs and for treating microvascular diseases.

### SUMMARY OF INVENTION

Superactivated platelet (SAP) aggregation in a patient is inhibited by administering to a subject in need of such inhibition an effective amount of a composition which comprises as active ingredients a stilbene, a flavonol, and μ-opioid receptor antagonist. A preferred stilbene is resveratrol, a preferred flavonol is quercetin, and a preferred μ-opioid receptor antagonist is naltrexone.

The stilbene, the flavonol, and the μ-opioid antagonist preferably are present in the composition in a respective mol ratio in the range of about 0.1:0.1:1 to about 10:10:50.

Inhibition of superactivated platelet (SAP) aggregation is useful for treating microvascular diseases with abnormal platelet function such as primary open-angle glaucoma (POAG), Alzheimer's disease, dementia, arthritis, cardiovascular disease, deep vein thrombosis, pulmonary embolism, diabetes, fibromyalgia, heart disease, lupus, myocardial infarction, scleroderma, Sjögren's Syndrome, stroke, and the like.

Inhibition of platelet aggregation can also reduce scar formation following surgical incisions. For this purpose platelet inhibition would be applicable, including but not limited, to the following: plastic surgery, ocular surgery, thoracic surgery, and the like. Also, inhibition of platelet aggregation is useful in preventing thrombus formation in blood vessels leading to deep vein thrombus, pulmonary embolism and death.

Compositions embodying this invention are useful for preventing, treating or reversing disorders and conditions related to certain platelet abnormalities and blood diathesis in patients with microvascular diseases including Alzheimer's disease, arthritis, blood coagulation related diseases, cardiovascular disease, deep vein thrombosis, diabetes, fibromyalgia, heart disease, lupus, myocardial infarction, primary open angle glaucoma, scleroderma, stroke, thromboembolic diseases and the like.

These compositions are also useful for decreasing blood viscosity at low shear rates.

### BRIEF DESCRIPTION OF DRAWINGS

In the drawings,

FIG. 1 is a diagram of coagulation cascade showing the intrinsic coagulation pathway (microvascular injury) and the extrinsic coagulation pathway (microvascular tissue damage). RQN denotes combinatorial drug treatment with resveratrol, quercetin and naltrexone; Toll-4 denotes toll-like receptor 4; SAP denotes superactivated platelet; XII denotes Hageman factor; XI denotes plasma thromboplastin antecedent (PTA); IX denotes plasma thromboplastin component (PTC); X denotes Stuart-Prower factor; VIII denotes antihemophilic factor (AHF); PL denotes plasma membrane phospholipid; Ca⁺⁺ denotes calcium ions; TF denotes tissue factor; VII denotes Proconvertin; and XIII denotes fibrin-stabilizing factor (FSF). For VII and X-XIII the subscript “a” indicates activated form of factor.

FIG. 2A is a nailfold capillaroscopy image from a 68 year old male suffering from normal tension glaucoma (NTG).

FIG. 2B is a nailfold capillaroscopy image from a 74 year old female suffering from primary open angle glaucoma (POAG).

FIG. 2C is a nailfold capillaroscopy image from a 63 year old male suffering from normal tension glaucoma (NTG).

In FIGS. 2A-2C arrows identify locations of hemorrhages within the capillary bed.

FIG. 3 is a graphical representation of the Combination Index in POAG and Alzheimer's disease patients who have received a combination of reservatrol, quercetin and naltrexone, each at 1 μM concentration.

FIG. 4 is a histogram showing the effect of combined in vivo administration of resveratrol, quercetin and naltrexone to mice.

FIG. 5 is a histogram showing the results of a thrombin generation test in POAG patients and Alzheimer's disease patients.

FIG. 6 is a histogram presenting the results of an evaluation of resveratrol, quercetin and naltrexone efficacy in reducing SAPs as compared to known anti-platelet drugs.

FIG. 7 is a schematic illustration of targets and signaling pathways of resveratrol, quercetin, naltrexone, and known anti-platelet drugs.

### DESCRIPTION OF PREFERRED EMBODIMENTS

Superactivated platelets (SAPs) are prothrombogenic and are prevalent in afflictions and diseases that have microvascular components, such as primary open-angle glaucoma (POAG) and neurodegenerative diseases such as dementia, Alzheimer's disease, transient ischemic attacks, ischemic stroke, and the like. SAPs also negatively influence recruitment of endothelial progenitor cells (EPCs) and repair of damaged blood vessel endothelium, and are responsible for increased number of nailfold hemorrhages and hemorrhages at the optic disc in patients suffering from POAG.

It has now been found that the SAP population in a subject can be reduced by a combinatorial drug intervention strategy and provide treatment for microvascular diseases such as POAG, neurodegenerative diseases, scar formation, thromboembolic diseases, and the like.

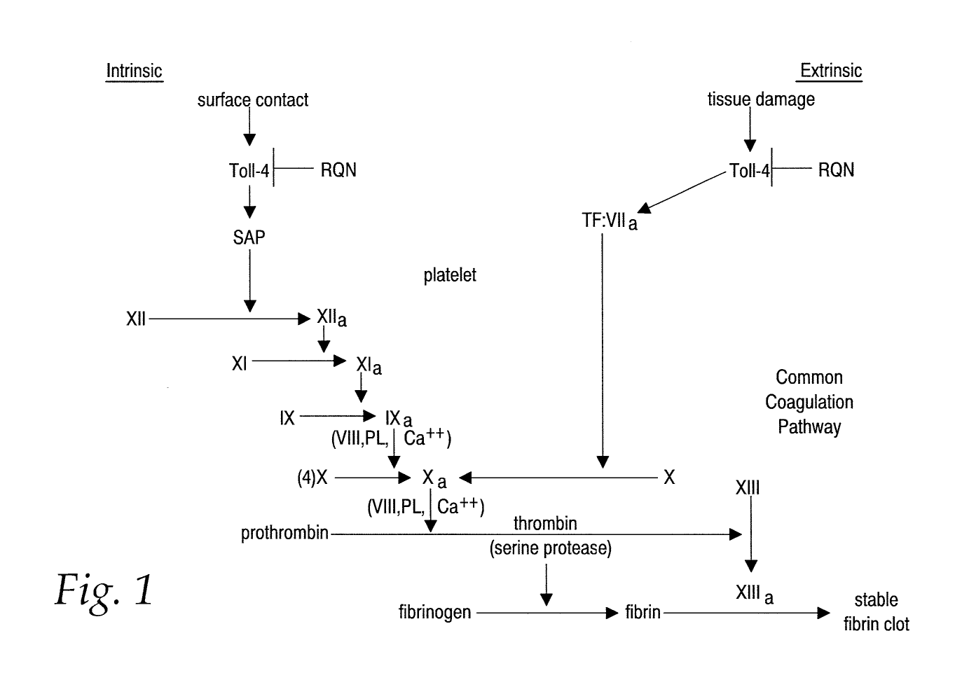

The present combinatorial drug strategy has been found to inactivate the toll-like receptor 4 (TLR4), thereby blocking the first step in the coagulation cascade for the intrinsic as well as the extrinsic coagulation pathway as shown in FIG. 1, in contradistinction to known anticoagulants such as aspirin, the nonsteroid antiinflammatory drugs (NSAIDs), dabigatran, and rivaroxaban. Specifically, aspirin and the NSAIDs target only platelets, dabigatran targets only thrombin, and rivaroxaban targets only Factor X.

Selected embodiments of the invention are shown and described herein. These embodiments are presented by way of example only. Variations, changes and substitutions will readily occur to those skilled in the art without departing from the invention.

Unless defined otherwise, all technical and scientific terms used herein have the same meaning as is commonly understood by one skilled in the art to which this invention belongs.

As used herein and in the claims, the singular form “a,” “an,” and “the” includes plural references unless the context clearly dictates otherwise.

The terms “treatment,” or “treating” or “ameliorating” as used in the specification and claims refer to an approach for achieving beneficial or desired results, including but not limited to a therapeutic benefit and/or a prophylactic benefit.

The term “therapeutic benefit” as used in the specification and the claims means eradication or amelioration of the underlying disorder being treated. A therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the present compositions may be administered to a subject at risk of developing a particular affliction or disease, or to a subject reporting one or more of the physiological symptoms of a disease even though a diagnosis of the disease may not have been made.

The term “antagonist” as used in the specification and claims refers to a compound having the ability to inhibit a biological function of a target protein or receptor. Accordingly, the term “antagonist” is defined in the context of the biological role of the target protein or receptor.

The term “effective amount” or “therapeutically effective amount” refers to that amount of composition described herein that is sufficient to achieve the intended effect. The effective amount may vary depending upon the intended application or the subject and disease condition being treated, e.g., the weight and age of the subject, the severity of the disease condition, the manner of administration and the like, which can be determined readily by one skilled in the art. This term also applies to a dose that induces a particular response, e.g., reduction of platelet adhesion. The specific dose may vary depending on the particular compounds that constitute the composition, the dosing regimen to be followed, timing of administration, and the physical delivery system in which the composition is carried.

The “effective amount” or “therapeutically effective amount” may be determined by using methods known in the art such as the NFkB-Luciferase Reporter Mice Assay, the Enzyme-Linked Immunosorbent Assay (ELISA), and the like. An increase in circulating IL-6 is indicative of enhanced TLR4 expression by the platelets, thus monitoring of serum IL-6 levels can also be utilized to arrive at an effective amount of the present compositions to be administered to a particular patient.

The term “pharmaceutically acceptable excipient” as used in the specification and claims includes any and all solvents, dispersion media, coatings, antibacterial and antifungal agents, isotonic and absorption retarding agents, and the like. The use of such agents and media for pharmaceutically-active substances is well known in the art. Supplementary active ingredients can also be incorporated into the compositions described herein.

“Subject” refers to an animal, such as a mammal, for example a human. The methods described and claimed herein can be useful in both human therapeutics and veterinary applications. In some embodiments the patient is a mammal, and in some embodiments the patient is a human.

The term “unit dosage form” as used in the specification and claims refers to physically discrete units suitable as unitary dosages for human subjects and animals, each unit containing a predetermined quantity of active material calculated to produce the desired therapeutic effect in association with the required pharmaceutical diluent, carrier, or vehicle. The specifications for the novel unit dosage forms of this invention are dictated by and directly dependent on (a) the unique characteristics of the active material and the particular therapeutic effect to be achieved, and (b) the limitations inherent in the art of compounding such active material for therapeutic use in humans and animals, as disclosed in detail in the specification, these being features of the present invention. Examples of suitable unit dosage forms in accord with this invention are tablets, capsules, pills, powder packets, granules, wafers, sachets, suppositories, segregated multiples of any of the foregoing, and other forms as herein described.

The designation “μM,” as used herein, denotes the micromolar concentration (10⁻⁶ mol/L) of the indicated compound, e.g., the stilbene, the flavonol, the μ-opioid, or the like, available for contact with platelets.

Compositions containing as active ingredients a stilbene or any of its biologically active derivatives or metabolites, a flavonol or any of its biologically active derivatives or metabolites, and a μ-opioid receptor antagonist or any of its biologically active derivatives or metabolites inhibit aggregation of superactivated platelets. The stilbene, the flavonol, and the μ-opioid receptor antagonist preferably are present in these compositions in a respective mol ratio in the range of about 0.1:0.1:1 to about 10:10:50.

Also suitable biologically active derivatives of the foregoing are the covalently binding fluorosulfonyl (FO₂S—) and fluorosulfonyloxy (FO₂SO—) derivatives of the stilbene, the flavonol, or the μ-opioid receptor antagonist. Such derivatives can be prepared as described in Dong et al., Angew. Chem. Int. Ed., 2014, vol. 53, pp. 9430-9448, and can serve by targeting active serine, tyrosine, threonine, lysine, cysteine or histidine residues.

Suitable stilbenes for the present compositions are resveratrol (3,5,4′-trihydroxy-trans-stilbene), α,β-dihydroresveratrol (3,4′,5-trihydroxybibenzyl), pterostilbene (3′,5′-dimethoxy resveratrol), pinosylvin (3′,5-dihydroxy-trans-stilbene), piceatannol (3,5,3′,4′-tetrahydroxy-trans-stilbene), and the like. Preferred stilbene is resveratrol.

Illustrative covalently binding biologically active derivatives of stilbenes are 3,5-dihydroxy-4-fluorosulfonyl-trans-stilbene, 3,5-dihydroxy-4-fluorosulfonyloxy-trans-stilbene, 3,4′-dihydroxy-5-fluorosulfonyl-trans-stilbene, 3,4′-dihydroxy-5-fluorosulfonyloxy-trans-stilbene, and the like.

Suitable flavonols are quercetin (3,3′,4′5,7-pentahydroxy-2-phenylchromen-4-one), 3-hydroxyflavone, azaleatin, fisetin, galangin, gossypetin, kaempferide, kaempferol, isorhamnetin, morin, myricetin, natsudaidain, pachypodol, zhamnazin, zhamnetin, and the like. Preferred flavonol is quercetin.

Illustrative covalently binding biologically active derivatives of flavonols are 3,4′,5,7-tetrahydroxy-3′-fluorosulfonyl-2-phenylchromen-4-one, 3,4′,5,7-tetrahydroxy-3′-fluorosulfonyloxy-2-phenylchromen-4-one, and the like.

Suitable μ-opioid receptor antagonists are naltrexone (17-(cyclopropylmethyl)-4,5α-epoxy-3,14-dihydroxymorphinan-6-one), naloxone, methylnaltrexone, naloxegol, alvimopan, and the like. Preferred μ-opioid receptor antagonist is naltrexone.

Illustrative covalently binding biologically active derivatives of μ-opioid receptor antagonists are 17-(cyclopropylmethyl)-4,5α-epoxy-3-hydroxy-14-fluorosulfonyl-morphinan-6-one, 17-(cyclopropylmethyl)-4,5α-epoxy-3-hydroxy-14-fluorosulfonyloxy-morphinan-6-one, and the like.

The compositions of the present invention can be administered in such oral dosage forms as tablets, capsules (each of which includes sustained release or timed release formulations), pills, dermal patches, powders, granules, elixirs, tinctures, suspensions, syrups and emulsions. Likewise, they may also be administered in intravenous (bolus or infusion), intraperitoneal, topical (e.g., ocular eyedrop), subcutaneous, intramuscular or transdermal (e.g., patch) form, all using forms well known to those of ordinary skill in the pharmaceutical arts.

The dosage regimen utilizing the compounds of the present invention is selected in accordance with a variety of factors including type, species, age, weight, sex and medical condition of the patient; the severity of the condition to be treated and the route of administration; the renal and hepatic function of the patient; and the particular composition employed. An ordinarily skilled physician, veterinarian or clinician can readily determine and prescribe the effective amount of the drug required to prevent, counter or arrest the progress of the condition.

The amount of active ingredients to be administered depends on the age, weight of the patient, the particular condition to be treated, the frequency of administration, and the route of administration.

The daily dose range for the stilbene, such as resveratrol, preferably is about 2 milligrams to about 300 milligrams, for the flavonol, such as quercetin, preferably is about 1 milligram to about 150 milligrams, and for the μ-opioid, such as naltrexone, is about 0.5 milligrams to about 300 milligrams. The daily dose can be administered as a single dose or in 3 or 4 divided doses. Veterinary dosages correspond to human dosages with the amounts administered being in proportion to the weight of the animal as compared to adult humans.

### Example 1: Isolation and Cytometry of SAP

A venipuncture sample of 10 mL of blood is obtained from each patient. Of the 10 mL, a 3 mL aliquot of blood is added to a tube containing 0.5 mL of acid-citrate-dextrose solution (38 mM citric acid, 75 mM sodium citrate, 135 mM glucose) to prevent clotting. The aliquot is diluted with 5 mL of buffered saline glucose citrate solution (129 mM NaCl, 13.6 mM sodium citrate, 11.1 mM glucose, 1.6 mM KH₂PO₄, 8.6 mM NaH₂PO₄, pH 7.3) and centrifuged. After centrifuging at 1100 rpm for 10 minutes at room temperature, platelet rich plasma (PRP), which is the upper layer, is removed. The PRP is transferred to a new tube, leaving about 0.5 mL above the buffy coat layer. The PRP-containing tube is incubated with 10 μL of thrombin and convulxin, two agonists that activate the platelets, for five minutes. Platelets are then treated with biotinylated fibrinogen and stained with the following fluorophores: APC-streptavidin (APC-SA), FITC-PAC1, and PE-anti-CD41. PE-anti-CD41 recognizes the platelet-specific, transmembrane protein CD41. FITC-PAC1 identifies activated platelets by recognizing GPIIaIIIb which increases its expression at the surface upon activation. APC-SA recognizes superactivated platelets that more readily bind the biotinylated fibrinogen to their surface.

All fluorophores are excited at 488 nm by argon laser of the flow cytometer. Their emission spectra are well separated, thereby allowing simultaneous, multi-color immunofluorescence measurements. Table 2, below, describes each fluorophore and the specific cell type it identifies.

TABLE 2
Platelet Fluorochromes

| | | Protein profile | Flurophores to detect |
|---|---|---|---|
| Platelets | Resting | CD41⁺/SB-Fibrinogen⁻/PAC1⁻ | PE-CD41 |
| | Activated | CD41⁺/SB-Fibrinogen⁻/PAC1⁺ | PE-CD41/FITC-PAC1 |
| | SAPs | CD41⁺/SB-Fibrinogen⁺/PAC1⁻ | PE-CD41/APC-SA |

Each sample is analyzed by a Beckman Coulter CyAn flow cytometer (Beckman Coulter, Fullerton, Calif.). Results are analyzed by the Summit program (Beckman Coulter). Data analysis is conducted for SAPs after flow cytometry using the Summit program to determine the relative percentages of each of these groups per sample. The SAPs are expressed as a percentage of the total number of events in the sample.

### Example 2: SAP Inhibition by Combinatorial Drug Treatment

Isolation and flow cytometry of SAPs was performed according to the procedure described in Example 1, above.

Aliquots of platelet rich plasma (PRP) were incubated with aqueous saline solutions of resveratrol only, quercetin only, naltrexone only, and with an admixture of resveratrol, quercetin and naltrexone (RQN) for a time period of 30 minutes at 37° C. Thereafter the obtained PRP samples were irradiated by an argon laser at 635 nm. Multi-color immunofluorescence was noted, and analyzed as described in Example 1, above. The obtained results are shown in Table 3, below. These data have been normalized relative to the Control (platelets incubated with thrombin and convulxin for five minutes).

TABLE 3
SAP Inactivation

| | % SAPs | % Reduction in SAPs |
|---|---|---|
| Control | 100 | 0 |
| resveratrol, 10 μM | 53.4 | 46.6 |
| quercetin, 10 μM | 22.2 | 77.8 |
| naltrexone, 50 μM | 47.8 | 52.2 |
| resveratrol (10 μM), quercetin (10 μM) | 6.4 | 93.6 |
| and naltrexone (50 μM) | | |

Data in Table 3, above, demonstrate a synergistic effect in SAP reduction by the combination of resveratrol, quercetin, and naltrexone.

### Nailfold Capillaroscopy

Nailfold capillaroscopy has been used since the 1970's to study a variety of diseases associated with vascular dysfunction, e.g., scleroderma and rheumatoid arthritis [Redisch W, Messina E J, McEwen C. Capillaroscopic observations in rheumatic diseases. Ann Rheum Dis 1970, 29:244-253]. A number of studies have supported these initial findings and more recently, have also identified hemorrhages in the nailfolds of primary open-angle glaucoma (POAG) patients [Park H Y, Park S H, O H Y S, Park C K. Nail bed hemorrhage: a clinical marker of optic disc hemorrhage in patients with glaucoma. Arch Ophthalmol 2011, 129:1299-1304; Begg I S, Drance S M, Sweeney V P. Ischemic optic neuropathy in chronic simple glaucoma. Br J Ophthalmol 1971, 55:73-90; Knepper P A, Norkett W M, Green K A, Wanderling C, Kuprys P V, Giovingo M, Tanna A P, Pasquale L R. Microvascular disease in glaucoma. In: Knepper P A, Samples J R (eds): Glaucoma Research and Clinical Advances: 2016 to 2018. Amsterdam, Kugler, 2016].

### Example 3: Inhibition of Hemorrhages in POAG Patients

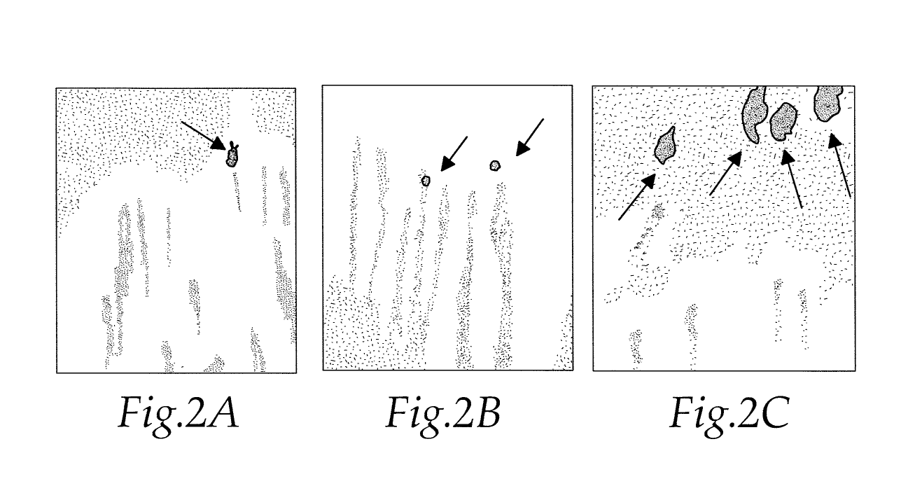

Nailfold capillary video microscopy was performed on the fourth and fifth digit of the non-dominant hand of the patient using a JH-1004 capillaroscope. High tension POAG (n=173), normal tension POAG (NTG) (n=28), and age-matched controls (n=138). The skin was made transparent with cedar wood oil and magnified by 275λ by the capillaroscope. Subjects were excluded based on previous medical history including connective tissue diseases (e.g., arthritis), autoimmune disorders (e.g., Sjögren's), malignancies and blood diathesis. Videos were taken of the nailfold and began at the medial side of the cuticle and ended at the lateral side. All videos underwent analysis by two masked observers for avascular areas, dilated capillaries, and hemorrhages. Examples of these microvascular events are illustrated in FIGS. 2A-C. An ANOVA was applied to each of the analyzed categories. A Dunnett's test was used when appropriate.

The observations are summarized in Table 4, below.

TABLE 4
Comparison of Nailfold Capillaroscopy of Various
Glaucoma and Glaucoma Suspect Patients

| | | | | | | Nailfold Dilated Capillaries | | | | | | | |
|---|---|---|---|---|---|---|---|---|---|---|---|---|---|
| | | | | | | (>50 μm) | | | | Nailfold Avascular Zones (>200 μm) | | | |
| | | Nailfold Hemorrhages | | | | With | | | | With | | | |
| | | With | | Events per | | Dilated | | Events per | | Avascular | | Events per | |
| | | Hemorrhages | | 100 | | Capillaries | | 100 | | Zones | | 100 | |
| Cohort | n | n | % | Capillaries | Significance | n | % | Capillaries | Significance | n | % | Capillaries | Significance |
| Control | 138 | 30 | 21.7 | 0.41 | N/A | 41 | 29.7 | 0.61 | N/A | 5 | 3.6 | 0.03 | N/A |
| POAG | 173 | 150 | 86.7 | 1.84 | p < 0.0005 | 83 | 48.0 | 0.95 | ns | 18 | 10.4 | 0.10 | ns |
| NTG | 28 | 23 | 82.1 | 3.71 | p < 0.0005 | 19 | 67.9 | 1.55 | ns | 12 | 41.4 | 0.49 | p < 0.0005 |

A statistical difference is seen between the normalized (per 100 capillaries) number of microvascular events between each of the groups compared to controls: POAG (p<0.0005) and NTG (p<0.0005). Notably 87% and 82% of POAG and NTG patients, respectively, displayed nailfold hemorrhages, compared to 21% of controls. Nailfold avascular zones were also more common in NTG patients compared to controls (p<0.0005): 4% of controls compared to 43% of NTG patients. No significance of dilated capillaries compared to controls was observed in any group.

The hemorrhages were analyzed for age (bright red suggesting a new event or dark brown suggesting hemosiderin), size (scaled accordingly), location (at the apex of the capillary loop or not) and patency (if blood was free flowing). A statistically significant difference in new microvascular events was observed when POAG and NTG patients were compared (p<0.001). Intravascular hemorrhages were more likely to occur in NTG patients suggesting a blood thrombotic event whereas extravascular hemorrhages were more common in POAG patients suggesting an injury to the capillary endothelium. Fifty-four percent of the new microvascular events observed in NTG patients were intravascular while only nine percent of POAG patients had intravascular events. The observations are summarized in Table 5, below.

TABLE 5
Intravascular vs. Extravascular Occurrences
in New Microvascular Events

| | | Control | POAG | NTG |
|---|---|---|---|---|
| Subjects (n) | | 9/138 | 86/176 | 7/28 |
| Fresh* | Intravascular | 1 | 7 | 7 |
| | Extravascular | 9 | 64 | 6 |

*A fresh hemorrhage is bright red blood which occurs either inside (intravascular) or outside (extravascular) a capillary.

### Example 4: Hemorrhages in Alzheimer's Disease (AD) Patients

Nailfold capillary beds were examined in patients with AD with mild cognitive impairment (MCI) as well as POAG and controls. Subjects were recruited from five sites after Institutional Review Board approval. Inclusive criteria were 70 to 90 years of age. Exclusion criteria were connective tissue disease and blood diathesis. Video microscopy was performed on AD (n=10) with MCI subjects with a global clinical dementia rating ≥0.5, POAG (n=56) patients and controls (n=46). All controls had IOP<21 mm Hg OU and a cup-disc ratio of less than 0.6. All POAG patients had manifest visual field loss on standard automated perimetric tests. All AD with MCI patients denied a history of glaucoma. Microvascular events were documented by video microscopy using a JH-1004 capillaroscope at 280× magnification on the subject's fourth and fifth finger of the non-dominant hand and characterized by masked observers. The observations are summarized in Table 6, below.

TABLE 6
Comparison of Nailfold Capillaroscopy of Control, POAG, and AD Patients

| | | | | Nailfold Dilated | | Nailfold Avascular Zones | |
|---|---|---|---|---|---|---|---|
| | | Nailfold Hemorrhages | | Capillaries (>50 μm) | | (>200 μm) | |
| | | Events per | | Events per | | Events per | |
| | | 100 | | 100 | | 100 | |
| Cohort | n | Capillaries | Significance | Capillaries | Significance | Capillaries | Significance |
| Control | 46 | 0.42 | | 0.49 | | 0.02 | |
| POAG | 56 | 2.06 | p < 0.001 | 1.02 | p = 0.1 | 0.17 | p = 0.3 |
| AD | 10 | 2.41 | p < 0.001 | 0.73 | p = 0.8 | 0.38 | p = 0.1 |

Nailfold hemorrhages were noted in 100% of AD/MCI patients, 86% of POAG patients compared to 24% of controls. The mean number of hemorrhages per 100 capillaries in Alzheimer's disease patients was 2.41±2.3 (p<0.001 compared to control subjects), in POAG patients was 2.06±2.0 (p<0.001 compared to control subjects) and 0.42±0.8 in controls. Dilated capillaries were present in 60% of Alzheimer's disease patients, 45% of POAG patients and 35% of control subjects (p=0.2). Avascular zones were observed in 30% Alzheimer's disease, 13% POAG, and 4% controls (p=0.1).

As noted above, both AD with MCI and POAG patients had significantly more NF hemorrhages compared to control patients, indicating that microvascular abnormalities exist in these patients. Since the NF hemorrhages are transient biomarkers lasting less than 7 days, the outcomes of therapeutic interventions to prevent the NF hemorrhages or treatments geared at Alzheimer's disease can be readily determined by video microscopy. The etiology of these peripheral microvascular events indicates microvascular disease in AD and POAG patients.

TABLE 7
Determination of Dose Ranges

| | | | | Dose Range⁴ at Concentration | | |
|---|---|---|---|---|---|---|
| Compound | Molar Mass | Bioavailability | Half-life | 0.1 μM | 10 μM | 50 μM |
| Resveratrol | 228.25 g/mol | 5%¹ | 1-3 hr | 2.28 mg | 228 mg | N/A* |
| Quercetin | 302.24 g/mol | 11%² | 16.8 hr | 1.37 mg | 137 mg | N/A* |
| Naltrexone | 341.40 g/mol | 33%³ | 4 hr | 0.517 mg | 5.17 mg | 258 mg |

*N/A, not applicable.
¹Reference: Poulsen M M, Vestergaard P F, Clasen B F, Radko Y, Christensen L P, Stodkilde-Jorgensen H, Moller N, Jessen N, Pedersen S B, Jorgensen J O. High Dose resveratrol supplementation in obese men: an investigator initiated, randomized, placebo-controlled clinical trial of substrate metabolism, insulin sensitivity, and body composition. Diabetes 2013; 62: 1186-1195.
²Graefe E, Wittig J, Mueller S, Riethling A, Uehleke B, Drewelow B, Pforte H, Jacobasch G, Derendorf H, Veit M. Pharmacokinetics and bioavailability of quercetin glycosides in humans J Clin Pharmacol 2001; 41: 492-499.
³Verebey K, Volavka J, Mule S J, Resnick R B. Naltrexone: disposition, metabolism, and effects after acute and chronic dosing. Clin Pharmacol Ther 1976; 20: 315-328.
⁴Administered amount to achieve indicated concentration of active compounds in patient's plasma, assuming a blood volume of 5 liters.

### Example 5: Hemorrhages and Reduction by RQN

In order to test the efficacy of the resveratrol, quercetin and naltrexone combination (RQN) in preventing hemorrhages, video microscopy was performed on ten-week-old male C57/BL6 mice using a JH-1004 capillaroscope on the front foot nailfold beds under isoflurane anesthesia. Mice were given resveratrol (5 μM), quercetin (5 μM), and naltrexone (10 μM) by 20 μL intraperitoneal injections at 1 and 3 hours prior to a thrombin injection. Thrombin (15 units) was injected intraperitoneal using a 27 gage needle in control (n=5). The observed results are compiled in Table 8, below.

TABLE 8
Comparison of Nailfold Hemorrhages
in Control and RQN-Treated Mice

Hemorrhages

| Cohort | Hemorrhages/100 capillaries | % with Hemorrhages |
|---|---|---|
| Control | 18.2 ± 10 | 100% |
| Treated | 3.0 ± 7 (p < 0.02) | 80% |

The above data show the efficacy of RQN in ameliorating nailfold hemorrhages.

The RQN admixture targets the innate immune toll-like receptor (TLR4) and minimizes hemorrhages by blocking both the intrinsic and extrinsic blood coagulation pathways. The combination of RQN is the only anti-platelet pharmaceutical composition known to act independently of cyclooxygenase pathway used by aspirin and other NSAID drugs. This in vivo example demonstrates the use of RQN in treating microvascular disease and the use of an anti-platelet pharmaceutical composition to prevent or at least minimize the occurrence of microhemorrhages.

### Example 6: Synergistic Effects of Resveratrol, Quercetin, and Naltrexone

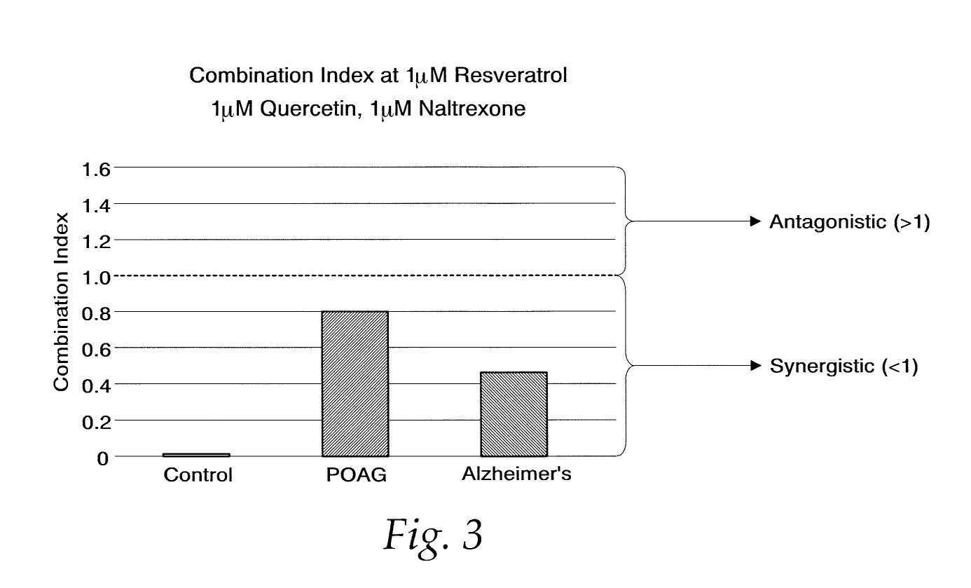

The Chou-Talalay combination index theorem was used to determine synergistic drug effects. The theorem is based on the median-effect equation to provide a common link between a single entity and multiple entities. A value <1 indicates synergism, =1 indicates additivity, and >1 indicates antagonism. Control (combination index=0.015; n=6), primary open angle glaucoma (POAG) (combination index=0.79; n=6), and Alzheimer's disease (combination index=0.48; n=6) cohorts were determined. Using 100-microliter aliquots of aqueous saline solutions it was found that at 1 μM concentration resveratrol, quercetin, and naltrexone act in a synergistic manner to reduce the amount of SAPs. This synergism allows for administration of a low dose of each of the compounds as opposed to a much higher dose of one compound. This also allows all three compounds to be administered at levels far below potentially harmful doses. The synergism of resveratrol, quercetin, and naltrexone found in control, POAG and Alzheimer's disease patients is shown in FIG. 3.

### Example 7: Example of Synergism in Normal, POAG, and Alzheimer's Disease Patients

Resveratrol, quercetin and naltrexone were tested in vitro using 100-microliter assay solutions with (A) a control subject platelets and (B) a POAG subject platelets. A marked reduction in SAPs at 1 μM resveratrol, 1 μM quercetin, 1 μM naltrexone in both the control subject platelets and the POAG patient's platelets was observed, representing a synergistic decrease as compared to the compounds alone. The observations are summarized in Table 9, below.

TABLE 9
Comparison of SAP in Response to Varying Doses of Resveratrol,
Quercetin, and Naltrexone Alone or in Combination
A)

| Control Patient | | | | | Control Patient | | | | |
|---|---|---|---|---|---|---|---|---|---|
| 68 year old Caucasion Female | | | | | 68 year old Caucasion Female | | | | |
| Cup/disc ratio 0.3/0.3. IOP 17,17 mmHG | | | | | Cup/disc ratio 0.3/0.3. IOP 17,17 mmHG | | | | |
| SAP Profile | | | | | SAP Profile | | | | |
| | | | | SAP | | | | | SAP |
| R (uM) | Q (uM) | N (uM) | SAP % | % Change | R (uM) | Q (uM) | N (uM) | SAP % | % Change |
| 0 | 0 | 0 | 36.29% | | 1 | 1 | 1 | 14.86% | −59.05% |
| 1 | 0 | 0 | 32.20% | −11.27% | 5 | 1 | 1 | 18.12% | −50.07% |
| 5 | 0 | 0 | 30.98% | −14.63% | 10 | 1 | 1 | 18.17% | −49.93% |
| 10 | 0 | 0 | 30.24% | −16.67% | 1 | 1 | 10 | 12.47% | −65.64% |
| 0 | 1 | 0 | 30.36% | −16.34% | 5 | 1 | 10 | 15.85% | −56.32% |
| 0 | 5 | 0 | 30.53% | −15.87% | 10 | 1 | 10 | 13.72% | −62.19% |
| 0 | 10 | 0 | 21.47% | −40.84% | 1 | 1 | 50 | 15.09% | −58.42% |
| 0 | 0 | 1 | 30.80% | −15.13% | 5 | 1 | 50 | 15.46% | −57.40% |
| 0 | 0 | 10 | 20.77% | −42.77% | 10 | 1 | 50 | 17.23% | −52.52% |
| 0 | 0 | 50 | 18.03% | −50.32% | 1 | 5 | 1 | 12.34% | −66.00% |
| 1 | 1 | 0 | 20.32% | −44.01% | 5 | 5 | 1 | 13.66% | −62.36% |
| 5 | 1 | 0 | 25.01% | −31.08% | 10 | 5 | 1 | 12.08% | −66.71% |
| 10 | 1 | 0 | 19.41% | −46.51% | 1 | 5 | 10 | 13.30% | −63.35% |
| 1 | 5 | 0 | 16.94% | −53.32% | 5 | 5 | 10 | 12.42% | −65.78% |
| 5 | 5 | 0 | 21.87% | −39.74% | 10 | 5 | 10 | 13.03% | −64.09% |
| 10 | 5 | 0 | 18.67% | −48.55% | 1 | 5 | 50 | 17.90% | −50.68% |
| 1 | 10 | 0 | 24.30% | −33.04% | 5 | 5 | 50 | 18.39% | −49.32% |
| 5 | 10 | 0 | 15.90% | −56.19% | 10 | 5 | 50 | 17.41% | −52.03% |
| 10 | 10 | 0 | 21.85% | −39.79% | 1 | 10 | 1 | 21.19% | −41.61% |
| 1 | 0 | 1 | 20.85% | −42.55% | 5 | 10 | 1 | 15.52% | −57.23% |
| 5 | 0 | 1 | 12.13% | −66.57% | 10 | 10 | 1 | 16.59% | −54.28% |
| 10 | 0 | 1 | 22.77% | −37.26% | 1 | 10 | 10 | 22.31% | −38.52% |
| 1 | 0 | 10 | 23.08% | −36.40% | 5 | 10 | 10 | 17.11% | −52.85% |
| 5 | 0 | 10 | 20.43% | −43.70% | 10 | 10 | 10 | 18.02% | −50.34% |
| 10 | 0 | 10 | 19.93% | −45.08% | 1 | 10 | 50 | 16.09% | −55.66% |
| 1 | 0 | 50 | 20.96% | −42.24% | 5 | 10 | 50 | 18.53% | −48.94% |
| 5 | 0 | 50 | 15.16% | −58.23% | 10 | 10 | 50 | 28.20% | −22.29% |
| 10 | 0 | 50 | 20.93% | −42.33% | | | | | |
| 0 | 1 | 1 | 17.59% | −51.53% | | | | | |
| 0 | 5 | 1 | 21.44% | −40.92% | | | | | |
| 0 | 10 | 1 | 11.67% | −67.84% | | | | | |
| 0 | 1 | 10 | 17.71% | −51.20% | | | | | |
| 0 | 5 | 10 | 15.03% | −58.58% | | | | | |
| 0 | 10 | 10 | 21.70% | −40.20% | | | | | |
| 0 | 1 | 50 | 15.65% | −56.88% | | | | | |
| 0 | 5 | 50 | 15.35% | −57.70% | | | | | |
| 0 | 10 | 50 | 12.41% | −65.80% | | | | | |

B)

| POAG Patient | | | | | POAG Patient | | | | |
|---|---|---|---|---|---|---|---|---|---|
| 75 year old African American Female | | | | | 75 year old African American Female | | | | |
| Cup/disc ratio 0.7/0.8. IOP 27,30 mmHG | | | | | Cup/disc ratio 0.7/0.8. IOP 27,30 mmHG | | | | |
| SAP Profile | | | | | SAP Profile | | | | |
| | | | | SAP | | | | | SAP |
| R (uM) | Q (uM) | N (uM) | SAP % | % Change | R (uM) | Q (uM) | N (uM) | SAP % | % Change |
| 0 | 0 | 0 | 56.88% | | 1 | 1 | 1 | 23.56% | −58.58% |
| 1 | 0 | 0 | 53.30% | −6.29% | 5 | 1 | 1 | 23.31% | −59.02% |
| 5 | 0 | 0 | 53.78% | −5.45% | 10 | 1 | 1 | 23.35% | −58.95% |
| 10 | 0 | 0 | 43.53% | −23.47% | 1 | 1 | 10 | 26.13% | −54.06% |
| 0 | 1 | 0 | 49.18% | −13.54% | 5 | 1 | 10 | 26.51% | −53.39% |
| 0 | 5 | 0 | 48.25% | −15.17% | 10 | 1 | 10 | 25.86% | −54.54% |
| 0 | 10 | 0 | 43.27% | −23.93% | 1 | 1 | 50 | 40.27% | −29.20% |
| 0 | 0 | 1 | 44.27% | −22.17% | 5 | 1 | 50 | 26.22% | −53.90% |
| 0 | 0 | 10 | 40.84% | −28.20% | 10 | 1 | 50 | 35.94% | −36.81% |
| 0 | 0 | 50 | 38.93% | −31.56% | 1 | 5 | 1 | 29.27% | −48.54% |
| 1 | 1 | 0 | 47.63% | −16.26% | 5 | 5 | 1 | 27.96% | −50.84% |
| 5 | 1 | 0 | 45.72% | −19.62% | 10 | 5 | 1 | 28.34% | −50.18% |
| 10 | 1 | 0 | 43.19% | −24.07% | 1 | 5 | 10 | 41.46% | −27.11% |
| 1 | 5 | 0 | 41.54% | −26.97% | 5 | 5 | 10 | 19.60% | −65.54% |
| 5 | 5 | 0 | 50.55% | −11.13% | 10 | 5 | 10 | 28.13% | −50.55% |
| 10 | 5 | 0 | 49.62% | −12.76% | 1 | 5 | 50 | 16.67% | −70.69% |
| 1 | 10 | 0 | 44.93% | −21.01% | 5 | 5 | 50 | 32.10% | −43.57% |
| 5 | 10 | 0 | 45.51% | −19.99% | 10 | 5 | 50 | 28.58% | −49.75% |
| 10 | 10 | 0 | 46.99% | −17.39% | 1 | 10 | 1 | 34.38% | −39.56% |
| 1 | 0 | 1 | 41.69% | −26.71% | 5 | 10 | 1 | 43.45% | −23.61% |
| 5 | 0 | 1 | 46.27% | −18.65% | 10 | 10 | 1 | 22.33% | −60.74% |
| 10 | 0 | 1 | 47.04% | −17.30% | 1 | 10 | 10 | 23.54% | −58.61% |
| 1 | 0 | 10 | 42.61% | −25.09% | 5 | 10 | 10 | 19.79% | −65.21% |
| 5 | 0 | 10 | 39.50% | −30.56% | 10 | 10 | 10 | 25.32% | −55.49% |
| 10 | 0 | 10 | 44.18% | −22.33% | 1 | 10 | 50 | 29.76% | −47.68% |
| 1 | 0 | 50 | 45.53% | −19.95% | 5 | 10 | 50 | 38.17% | −32.89% |
| 5 | 0 | 50 | 50.24% | −11.67% | 10 | 10 | 50 | 44.70% | −21.41% |
| 10 | 0 | 50 | 40.90% | −28.09% | | | | | |
| 0 | 1 | 1 | 45.02% | −20.85% | | | | | |
| 0 | 5 | 1 | 48.00% | −15.61% | | | | | |
| 0 | 10 | 1 | 43.87% | −22.87% | | | | | |
| 0 | 1 | 10 | 45.35% | −20.27% | | | | | |
| 0 | 5 | 10 | 43.06% | −24.30% | | | | | |
| 0 | 10 | 10 | 45.52% | −19.97% | | | | | |
| 0 | 1 | 50 | 45.24% | −20.46% | | | | | |
| 0 | 5 | 50 | 43.02% | −24.37% | | | | | |
| 0 | 10 | 50 | 44.87% | −21.11% | | | | | |

In Table 9 the combination index for synergism was found to be 0.023 for the control patient and 0.08 for the POAG patient, indicating strong synergistic effects. Separate administration of the compounds was not sufficient to achieve comparable SAP decreases at low doses.

### Example 8: In Vivo Trials

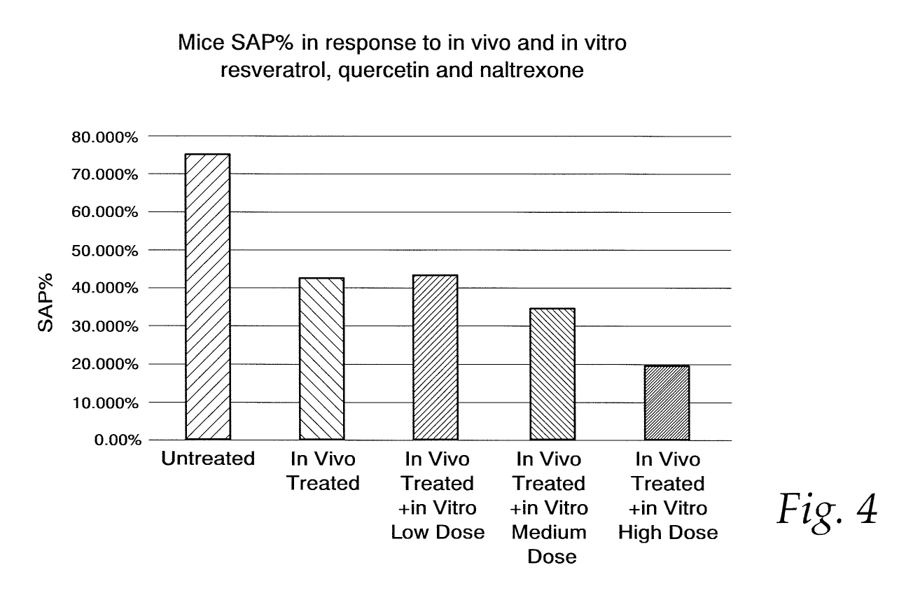

The in vivo effects of resveratrol, quercetin, and naltrexone (RQN) in combination was tested by an intraperitoneal (IP) injection in mice. 8-10 Week old mice received a 20 μL IP injection containing 5 μM resveratrol, 5 μM quercetin, and 10 μM naltrexone. SAP levels were found to be 75% in the untreated mice, and 42% in the treated mice. Blood from the treated mice was then collected and treated again with resveratrol, quercetin, and naltrexone in low (1 μM resveratrol, 1 μM quercetin, 1 μM naltrexone), medium (5 μM resveratrol, 5 μM quercetin, 10 μM naltrexone) and high (10 μM resveratrol, 10 μM quercetin, 50 μM naltrexone) concentrations. A 43% reduction in SAPs in the mice treated with 5 μM resveratrol, 5 μM quercetin, and 10 μM naltrexone was observed compared to the untreated mice. The observed results are shown in FIG. 4 and clearly demonstrate that systemic administration of resveratrol, quercetin, and naltrexone decreases SAP percentage. Blood samples from the mice systemically treated with resveratrol, quercetin, and naltrexone were further analyzed in order to evaluate the dose response in vitro. Further treatment of SAPs in vitro showed no further SAP reduction at low and medium doses, suggesting that the receptors were sufficiently saturated during the in vivo treatment.

### Example 9: Endothelial Progenitor Cells

Endothelial progenitor cells (EPCs) have been shown to play a vital role in angiogenesis and in the repair of damaged blood vessels. EPC levels are decreased in conditions that have microvascular abnormalities such as primary open-angle glaucoma (POAG) and Alzheimer's disease. Therefore, a treatment to increase the number and functionality of a patient's EPCs would reduce the symptoms of or cure the patient's condition. One method of achieving this increase in EPC counts is to perform an autologous transplant which involves collecting a blood sample from the patient, culturing the sample to enrich the EPC population, and transplanting the cells back into the patient. The transplanted EPCs allow for the repair of the damaged microvasculature. Previous studies have shown that autologous EPC transplant is an effective treatment method for advanced cardiovascular disease.

In particular, whole blood was collected from control subjects (n=4) via venipuncture, and separated via density gradient centrifugation using Lymphoprep (Stemcell Technologies, Vancouver, Canada). The peripheral blood mononuclear cell layer was then collected, resuspended in 5 ml of CFU-Hill Media (Stemcell Technologies), and plated onto a fibronectin-coated 6-well plate (Corning, Corning, N.Y.). After two days, the non-adherent cells, which are the endothelial progenitor cells (EPC), were removed and re-plated onto an 8-well chamber slide containing 5 ml of CFU-Hill Media. The cells were treated with 5 milliliters of RQN solution at low (1:1:1 μM), medium (5:5:10 μM), or high (10:10:50 μM) concentration on days 0 and 2. On the 5th day of culture, the plates were viewed for the presence of colony forming units (CFU). EPCs were identified in culture using dil-acetylated-LDL (endothelial cell marker) and FITC-ulex (fucose-residue marker) and through flow cytometry as CD34+/CD309+/CD133+ cells.

As can be seen in Table 10, below, resveratrol, quercetin, and naltrexone together increase the number of cultured EPCs.

TABLE 10
Isolation and Culture of Endothelial Progenitor Cells in the
Presence of Resveratrol, Quercetin, and Naltrexone (RQN)
(A)

| Treatment | CFU Count (n = 10) |
|---|---|
| Control | 10 ± 3.16 |
| EtOH Control | 10 ± 4.39 |
| Low Concentration RQN | 11 ± 3.13 |
| (1 μM, 1 μM, 1 μM) | (p = 0.6) |
| Medium Concentration RQN | 12.2 ± 4.49 |
| (5 μM, 5 μM 10 μM) | (p = 0.09) |
| High Concentration RQN | 13.1 ± 5.73 |
| (10 μM, 10 μM, 50 μM) | (p = .02) |

(B)

| Day 0 | Day 2 | Day 5 |
|---|---|---|
| 214 | 6647 | 27955 |

(A) Colony forming unit (CFU) count at Day 5 of culture. Compared to control groups, low, medium, and high concentrations of RQN gave an increase in CFU counts of 10%, 22%, and 31% respectively.
(B) EPC count as determined by flow cytometry. Numbers reported as CD34+/CD309+/CD133+ cells/10⁶ cells as determined by manual cell count (n = 8).

### Example 10: Safety Evaluation

Thrombin generation test, a clinical test to determine if a drug impedes blood clotting, was conducted as described below.

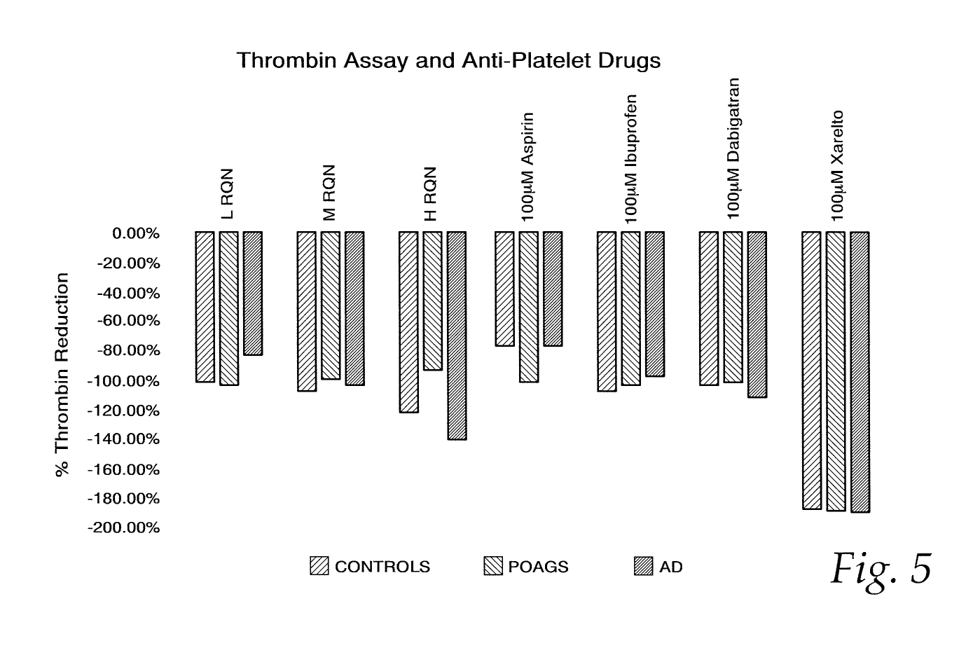

Resveratrol, quercetin, and naltrexone (RQN) were tested in vitro using a thrombin generation test to determine the effect of the RQN compositions on thrombin production. Low (L; 1 μM resveratrol, 1 μM quercetin, 1 μM naltrexone), medium (M; 5 μM resveratrol, 5 μM quercetin, 10 μM naltrexone), and high (H; 10 μM resveratrol, 10 μM quercetin, 50 μM naltrexone) concentrations were compared to known anti-platelet drugs. The results are shown in Table 11, below, and in FIG. 5. The RQN combination decreased thrombin in a manner similar to other commonly used anti-platelet drugs, further indicating the safety of the RQN combination.

TABLE 11
Comparison of Thrombin Reduction with Anti-Platelet Drugs

| | | Thrombin % Decrease | | |
|---|---|---|---|---|
| | | Control | POAG | Alzheimer's |
| Drug | Concentration | (n = 6) | (n = 6) | (n = 6) |
| Resveratrol, Quercetin, | 1 μM resveratrol, 1 μM | −101.27% | −103.38% | −84.61% |
| Naltrexone | quercetin, 1 μM naltrexone | | (p = 0.9)* | (p = 0.9) |
| Resveratrol, Quercetin, | 5 μM resveratrol, 5 μM | −108.75% | −101.16% | −103.41% |
| Naltrexone | quercetin, 10 μM naltrexone | | (p = 0.8) | (p = 0.9) |
| Resveratrol, Quercetin, | 10 μM resveratrol, 10 μM | −121.51% | −95.66% | −141.22% |
| Naltrexone | quercetin, 50 μM naltrexone | | (p = 0.6) | (p = 0.9) |
| Aspirin^{†} | 100 μM | −76.05% | −104.39% | −75.46% |
| | | | (p = 0.4) | (p = 0.9) |
| Ibuprofen^{†} | 100 μM | −106.45% | −101.37% | −95.08% |
| | | | (p = 0.9) | (p = 0.9) |
| Dabigatran^{†} | 100 μM | −101.97% | −100.45% | −109.55% |
| (Direct Thrombin Inhibitor) | | | (p = 0.9) | (p = 0.9) |
| Rivaroxaban^{†} | 1 μM | −183.06% | −184.65% | −186.56% |
| (Factor X Inhibitor) | | | (p = 0.1) | (p = 0.6) |

*p-values reported compared to treated controls.
^{†}The anti-platelet drug doses are comparable to routine clinical doses.

### Example 11: Efficacy Evaluation

The efficacy of resveratrol, quercetin, and naltrexone in preventing SAPs was evaluated.

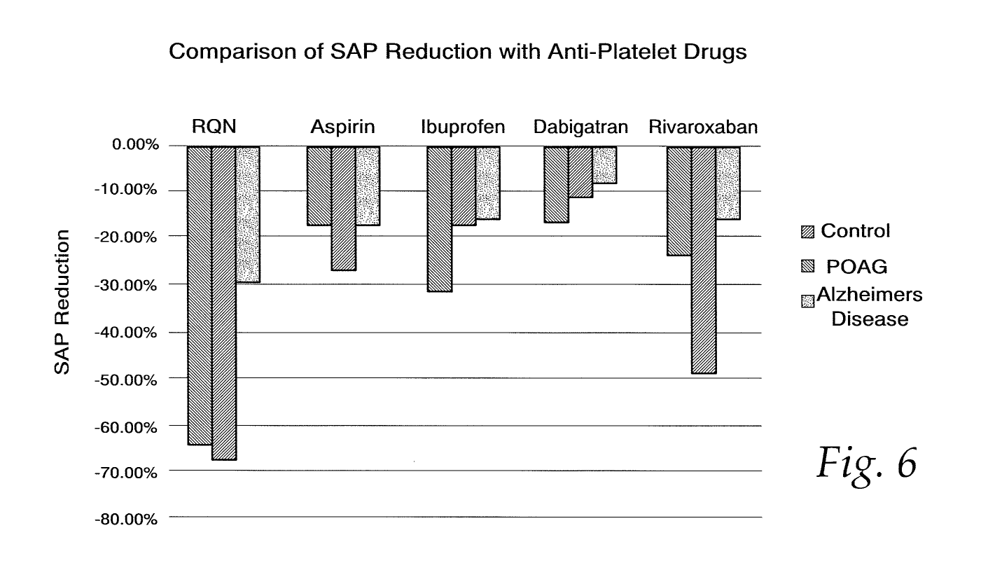

SAPs were analyzed in the presence of resveratrol, quercetin, and naltrexone and compared to known anti-platelet drugs. Student's t-test with Turkey Post Hoc correction was used to determine significance levels comparing untreated to drug treated samples. The appropriate doses for other anti-platelet drugs were established by routine clinical use. The results are shown in Table 12, below, and FIG. 6. Co-administration of resveratrol, quercetin and naltrexone reduced the SAP levels significantly more than the known common antiplatelet drugs.

TABLE 12
Comparison of SAP Reduction with Anti-Platelet Drugs

| | | SAP % Decrease | | |
|---|---|---|---|---|
| | | Control | POAG | Alzheimer's |
| Drug | Concentration | (n = 6) | (n = 6) | (n = 6) |
| Resveratrol, Quercetin, | 1 μM resveratrol, 1 μM | −63.68% | −66.85% | −29.61% |
| Naltrexone | quercetin, 1 μM naltrexone | (p < 0.001) | (p < 0.001) | (p < 0.01) |
| Aspirin | 100 μM | −17.00% | −26.69% | −17.32% |
| | | (p = 0.15/ins) | (p < 0.1/ins) | (p = 0.24/ins) |
| Ibuprofen | 100 μM | −31.40% | −16.84% | −15.30% |
| | | (p < 0.05) | (p = 0.3/ins) | (p = 0.19/ins) |
| Dabigatran (Pradaxa) | 100 μM | −0.16% | −11.21% | −8.09% |
| (Direct Thrombin Inhibitor) | | (p = 0.99/ins) | (p = 0.4/ins) | (p = 0.7/ins) |
| Rivaroxaban (Xarelto) | 1 μM | −23.38% | −48.73% | −15.63% |
| (Factor X Inhibitor) | | (p < 0.1/ins) | (p = 0.2/ins) | (p = 0.3/ins) |

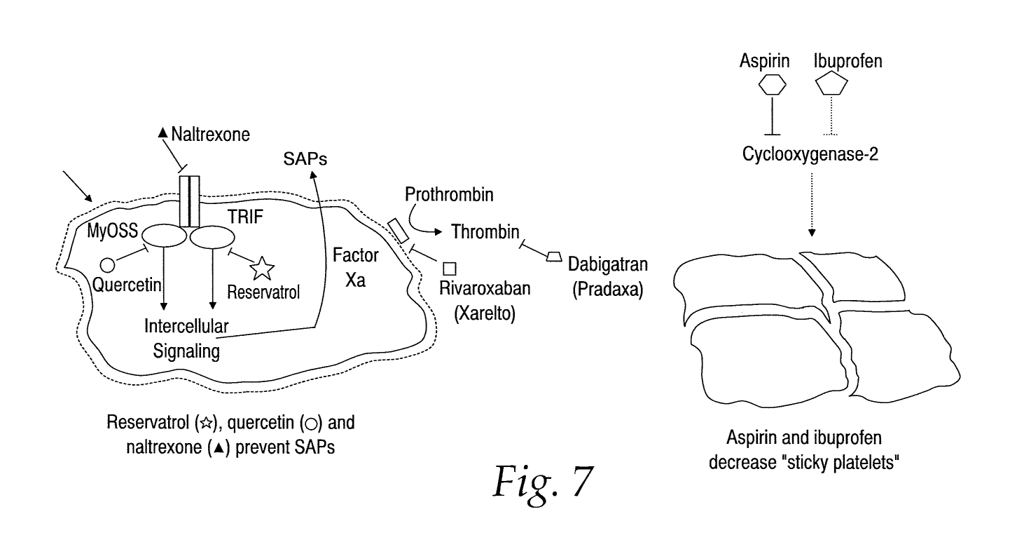

As illustrated by FIG. 7, resveratrol, quercetin, and naltrexone have a different target than existing anti-platelet drugs, and also act on a different signaling pathway.

The mechanism of resveratrol, quercetin, and naltrexone is different from that of existing anti-platelet drugs. Resveratrol, quercetin and naltrexone act together to prevent activation of an innate immune system receptor. Resveratrol, quercetin, and naltrexone together act concurrently on three legs of the TLR4 receptor—the TLR4 receptor itself, the MyD88 dependent pathway, and the MyD88 independent pathway—to inhibit subsequent intercellular signaling and superactivated platelet formation. Once a platelet is superactivated, it undergoes a phosphatidyl membrane flip, exposing negatively charged residues. This negative charge then serves as a platform for Factor Xa, which converts prothrombin to thrombin in the coagulation cascade. Rivaroxaban acts as a Factor Xa inhibitor, and dabigatran serves as a direct thrombin inhibitor. Aspirin and ibuprofen act on cyclooxygenase-2 to prevent “sticky platelets.” The combined administration of resveratrol, quercetin, and naltrexone represents a unique breakthrough therapy for platelet associated diseases. Resveratrol, quercetin, and naltrexone coact to suppress platelet activation, in marked contrast to existing drugs which are effective only after platelets have been activated.

This difference in mechanism has significant safety implications. Current anti-platelet medications such as aspirin increase the risk of gastrointestinal bleeding. Using aspirin in combination with clopidogrel or warfarin also increases the risk of upper gastrointestinal bleeding. Blockade of COX-1 by aspirin increases the gastric mucosal erosion. Aspirin causes an increased risk of cerebral microbleeds since they often occur prior to ischemic stroke or intracerebral hemorrhages, and Alzheimer's disease. In addition, current anti-platelet drugs can lead to uncontrolled bleeding and death. Aspirin and rivaroxaban effects on platelets are irreversible, whereas dabigatran can be reversed by praxbind. The combined anti-platelet effects of resveratrol, quercetin, and naltrexone can be reversed by an opiate analog, if necessary.

As evidenced by data in Table 12 and FIG. 6, current anti-platelet drugs do not significantly alter SAP production, except for rivaroxaban in POAG patients. However, the inhibition of the TLR4 receptor by the RQN combination, a receptor involved in the innate immune system, significantly decreases the number of SAPs.

### Example 12: Unit Dose Formulation

A uniform powder blend is formulated as a 10-gram batch for encapsulation or packaging in folded paper sachets using the amounts of active pharmaceutical ingredients (APIs) shown in Table 13, below.

TABLE 13
APIs in Exemplary Unit Doses

| | Dose | Batch |
|---|---|---|
| Formulation | (mg) | (g) |
| Resveratrol | 2.28 | 1.14 |
| Quercetin | 1.37 | 0.685 |
| Naltrexone | 5.17 | 2.585 |
| Mannitol | 9.98 | 4.99 |
| Sucralose | 0.8 | 0.4 |
| Colloidal silica dioxide | 0.2 | 0.1 |
| Magnesium stearate | 0.2 | 0.1 |
| | 20 | 10 |

One unit dose equivalent of the resulting powder blend is then encapsulated using a tabletop capsule filling machine and hard gelatin or hydroxypropyl-methylcellulose (HPMC) capsules.

The foregoing discussion and the examples are intended as illustrative and are not to be taken as limiting. Still other variants within the spirit and scope of the invention are possible and will readily present themselves to those skilled in the art.

1. A pharmaceutical composition which comprises a stilbene, a flavonol, and a μ-opioid receptor antagonist as active ingredients, together with a pharmaceutically acceptable excipient.

2. The composition in accordance with claim 1 wherein the stilbene, the flavonol, and the μ-opioid receptor antagonist are present in a respective mol ratio in the range of about 0.1:0.1:1 to about 10:10:50.

3. The pharmaceutical composition in accordance with claim 1 wherein the stilbene is resveratrol, the flavonol is quercetin, and the μ-opioid receptor antagonist is naltrexone.

4. A method of inhibiting superactivated platelet aggregation in a subject which comprises administering to the subject an effective amount of a composition containing as active ingredients a stilbene, a flavonol and a μ-opioid receptor antagonist.

5. The method in accordance with claim 4 wherein the stilbene, the flavonol, and the μ-opioid receptor antagonist are present in a respective mol ratio in the range of about 0.1:0.1:1 to about 10:10:50.

6. The method in accordance with claim 4 wherein the stilbene is resveratrol the flavonol is quercetin, and the μ-opioid receptor antagonist is naltrexone.

7. A method of blocking activation of coagulation cascade in a patient which comprises administering to the patient a coagulation cascade blocking amount of the pharmaceutical composition defined by claim 1.

8. A method of modulating the activation of toll-like receptor (TLR4) in a patient suffering from microvascular disease which comprises administering to the patient an effective amount of a pharmaceutical composition defined by claim 1.

9. A method of treating a subject suffering from a neurodegenerative disease which comprises administering to the subject an effective amount of a pharmaceutical composition defined by claim 1.

10. The method in accordance with claim 9 wherein the neurodegenerative disease is Alzheimer's disease.

11. The method in accordance with claim 9 wherein the neurodegenerative disease is dementia.

12. A method of treating a patient suffering from primary open-angle glaucoma which comprises administering to the patient an effective amount of the pharmaceutical composition defined by claim 1.

13. A method of treating a patient suffering from a thromboembolic disease which comprises administering to the patient an effective amount of the pharmaceutical composition defined by claim 1.

14. A method of preventing scar formation which comprises administering to a subject having received a soft tissue injury an effective amount of the composition defined by claim 1.
